(19) United States
(12) Patent Application Publication
(10) Pub. No.: US 2020/0037890 A1
(43) Pub. Date: Feb. 6, 2020

(54) METHOD AND APPARATUS FOR MEASURING BIO-SIGNAL USING RADAR

(71) Applicant: WRT LAB CO., LTD., Seoul (KE)

(72) Inventors: Sung Ho Cho, Seoul (KR); Jeong Woo Choi, Seoul (KR); Young Hyo Lim, Gyeonggi-do (KR); Yu Na Lee, Gyeonggi-do (KR)

(73) Assignee: WRT LAB CO., LTD., Seoul (KR)

(21) Appl. No.: 16/469,882

(22) PCT Filed: Dec. 13, 2017
(86) PCT No.: PCT/KR2017/014665

§ 371 Date: Oct. 14, 2019

(30) Foreign Application Priority Data
Dec. 14, 2016 (KR) 10-2016-0170138

(51) Int. Cl. A61B 5/024; A61B 5/00; A61B 5/0205; A61B 5/08

(52) U.S. Cl. CPC A61B 5/024; A61B 5/0004; G01S 13/88; A61B 5/08; A61B 5/0205

(57) ABSTRACT

Provided is a method and apparatus for measuring a bio-signal using a radar. An apparatus for measuring a bio-signal using a radar according to an embodiment of the present invention comprises: a first signal acquisition unit for acquiring, from a first radar, a first signal including a first bio-signal measured from a measurement subject, a second signal acquisition unit for acquiring, from a second radar, a second signal including both a second bio-signal and the first bio-signal measured from the measurement subject; a signal synchronization unit for synchronizing the first signal and the second signal and a bio-signal detection unit for calculating the difference between the synchronized first and second signals so as to eliminate the first bio-signal and detect the second bio-signal from the second signal.

### TECHNICAL FIELD

The present invention relates to a method and apparatus for measuring a bio-signal by using a radar. More particularly, the present invention relates to a method and apparatus for measuring a bio-signal such as a heartbeat signal by using a plurality of radars.

### BACKGROUND ART

Recently, research and development of a bio-radar for detecting breathing and heartbeat signals in a non-contact manner is underway.

However, in case of a bio-radar, since a subject to be measured is the human body, radio frequency (RF) transmission signal power that can be radiated is severely limited. In addition, a signal received from the human body is very weak, and thus it is very vulnerable to noise and interference from the surroundings.

Accordingly, a technique is provided where an impulse-radio ultra wide band (IR-UWB) radar (hereinafter, referred as “UWB radar”) is used to measure a bio-signal.

Herein, “ultra wide band (UWB)” is a radio technique that uses a frequency bandwidth of 500 MHz or more or using a broadband frequency of 25% or more, which is defined as the bandwidth of the signal versus the bandwidth of the signal, and the UWB is advantageous in range resolution, permeability, strong immunity against narrowband noise, and coexistence with other devices sharing a frequency.

UWB radars are radars on which such UWB technique is grafted on the radar, and can recognize surrounding environments by transmitting an impulse signal having a short duration with a broadband characteristic in the frequency domain, and by receiving a signal that is reflected from an object or person.

Due to such characteristics of UWB radars, research is being actively conducted to utilize UWB radars in various fields such as medical apparatuses for measuring breathing rate and heartbeat rate, portable radar apparatuses for rescuing people in a disaster scene, apparatuses for counting a number of people in a certain area, etc.

In an example, in Korean Patent Application Publication No. 10-2014-0106795, a “UWB-based contactless biometric signals tester” proposes a method of measuring a bio-signal of a breathing rate or heartbeat rate by using a UWB radar and providing a remote health management system by using the same.

Such a conventional technique is a method of converting a time axis into a frequency domain so as to extract a heartbeat rate from a radar signal, and detecting a heartbeat rate within a frequency domain of a typical heartbeat rate.

FIG. 1 is a view showing a graph of a result of measuring a bio-signal by using a conventional radar.

A graph of FIG. 1 is a result of converting a radar signal reflected from a subject to be measured into a frequency domain by using fast Fourier transform (FFT), and shows mainly a breathing signal, but a heartbeat signal is relatively weak.

Conventionally, a heartbeat signal is detected in a frequency range of a heartbeat rate by removing the breathing signal from a signal of a frequency domain.

However, such a conventional method extracts an average heartbeat rate during a certain time through frequency detection, but monitoring an interval of a real-time heartbeat waveform is difficult since a pulse of each heartbeat is not monitored in a time axis.

In addition, motions due to breathing are larger than motions due to a heartbeat, and thus accuracy is degraded when detecting a heartbeat waveform in real-time from a radar signal including both breathing and heartbeat signals by using a conventional method.

Accordingly, for patients with arrhythmia where an interval of heartbeat waveform changes in real time, or for a case other than arrhythmia patients when real-time period information of a heartbeat waveform is required, detecting a heartbeat signal by using the conventional method is difficult.

### DISCLOSURE

### Technical Problem

Accordingly, the present invention has been made keeping in mind the above problems occurring in the prior art, and an object of the present invention is to provide a method of detecting a real-time heartbeat signal from a radar signal including both breathing and heartbeat signals, and improving an accuracy thereof.

### Technical Solution

In order to accomplish the above object, according to an embodiment of the present invention, there is provided an apparatus for measuring a bio-signal by using a radar, the apparatus including: a first signal obtaining unit obtaining a first signal including a first bio-signal of a subject to be measured from a first radar; a second signal obtaining unit obtaining a second signal including both of first bio-signal and a second bio-signal of the subject to be measured from a second radar; a signal synchronizing unit synchronizing the first signal and the second signal; and a bio-signal detecting unit calculating a difference between the synchronized first and second signals, and determining the second bio-signal by removing the first bio-signal from the second signal, wherein the first radar is disposed at a set distance such that the first bio-signal is measured from the subject to be measured, and the second radar is disposed at a set distance such that both of the first bio-signal and the second bio-signal are measured.

In order to accomplish the above object, according to another embodiment of the present invention, there is provided an apparatus for measuring a bio-signal by using a radar, the apparatus including: a first signal obtaining unit obtaining a first signal including a first bio-signal of a subject to be measured from a first radar; a second signal obtaining unit obtaining a second signal including both of the first bio-signal and a second bio-signal of the subject to be measured from a second radar; a signal synchronizing unit synchronizing unit synchronizing the first signal and the second signal; and a bio-signal detecting unit calculating a difference between the synchronized first and second signals, and determining the second bio-signal by removing the first bio-signal from the second signal, wherein the first radar has a set gain such that the first bio-signal is measured from the subject to be measured, and the second radar has a set gain such that both of the first bio-signal and the second bio-signal are measured from the subject to be measured.

In order to accomplish the above object, according to an embodiment of the present invention, there is provided a method of measuring a bio-signal, wherein a bio-signal measuring apparatus measures a bio-signal by using a radar, the method including: (a) obtaining a first signal including a first bio-signal of a subject to be measured from a first radar, and obtaining a second signal including both of the first bio-signal and a second bio-signal of the subject to be measured from a second radar; (b) synchronizing the first signal and the second signal; and (c) calculating a difference between the synchronized first signal and second signals, and determining the second bio-signal by removing the first bio-signal from the second signal, wherein the first radar is disposed at a set distance such that the first bio-signal is measured from the subject to be measured, and the second radar is disposed in a set distance such that both of the first bio-signal and the second bio-signal are measured.

### Advantageous Effects

According to an embodiment of the present invention, a heartbeat rate, as well as a heartbeat waveform can be detected in real-time from a radar signal including both breathing and heartbeat signals.

It should be understood that the effects of the present invention are not particularly limited to those described above, and the present invention includes all effects that can be deduced from the detailed description of the invention or the configurations of the invention described in the claims.

### DESCRIPTION OF DRAWINGS

FIG. 1 is FIG. 1 is a view showing a result of measuring a bio-signal by using a conventional radar.

FIG. 2 is a view showing a configuration of a system for measuring a bio-signal by using a radar according to an embodiment of the present invention.

FIG. 3 is a view showing a block diagram of a bio-signal measuring apparatus according to an embodiment of the present invention.

FIG. 4 is a view showing a configuration of a system for measuring a bio-signal by using a radar according to another embodiment of the present invention.

FIG. 5 is a view showing a block diagram of a bio-signal measuring apparatus according to another embodiment of the present invention.

FIG. 6 is a view of a flowchart showing a process of measuring a bio-signal by using a radar according to an embodiment of the present invention.

FIG. 7 is a view of a flowchart showing a process of measuring a bio-signal by using a radar according to another embodiment of the present invention.

FIG. 8 is a view showing an experiment result of measuring a bio-signal by using a radar according to an embodiment of the present invention.

FIG. 9 is a view showing an experiment result of measuring a bio-signal by using a radar according to another embodiment of the present invention.

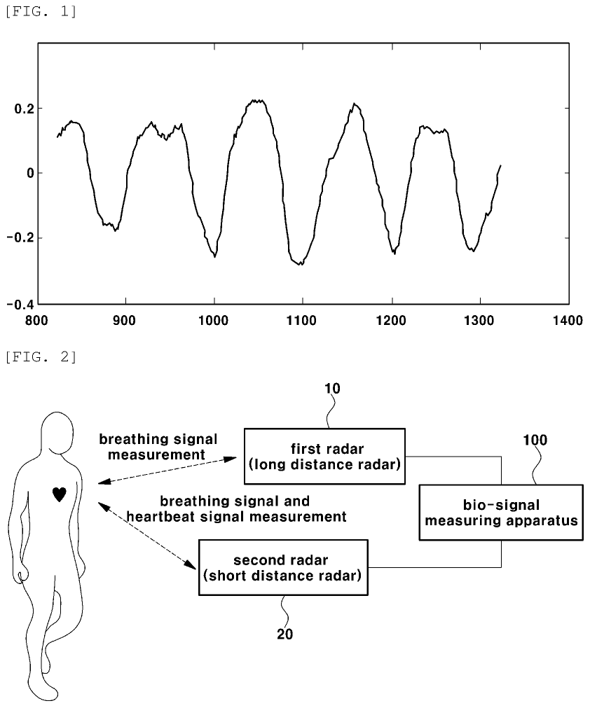

### MODE FOR INVENTION

Since the present invention may be modified in various forms, and may have various embodiments, the following exemplary embodiments are illustrated in the accompanying drawings, and are described in detail with reference to the drawings However, this is not intended to limit the present invention to specific embodiments, and the present invention should be construed to encompass various changes, equivalents, and substitutions within the technical scope and spirit of the invention. Like numbers refer to like elements throughout in the description of each drawing.

Hereinafter, various embodiments of the present invention will be described with reference to the accompanying drawings.

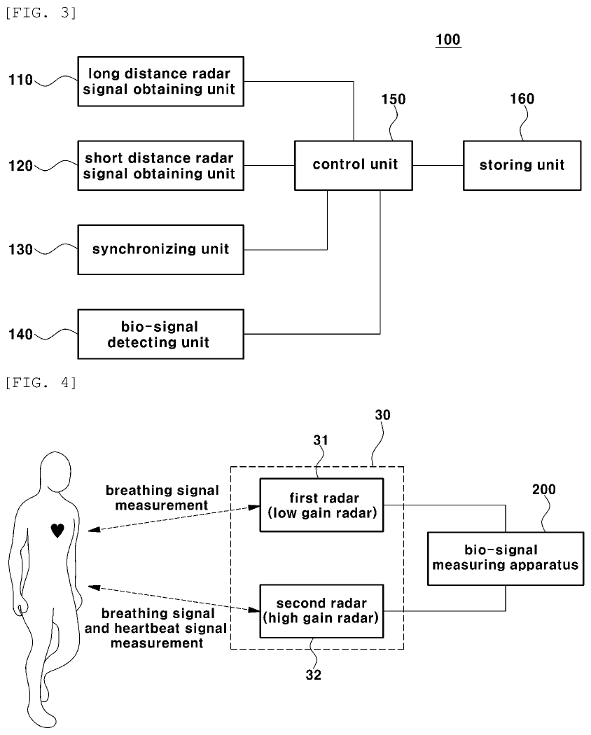

FIG. 2 is a view showing a configuration of a system for measuring a bio-signal by using a radar according to an embodiment of the present invention.

A system for measuring a bio-signal by using a radar according to an embodiment of the present invention may include a first radar 10, a second radar 20, and a bio-signal measuring apparatus 100.

For reference, bio-signals of a subject to be measured which are measured by using the present invention include various signals such as breathing, heartbeat and muscle relaxation and contraction signals. Hereinafter, an example will be described where a heartbeat signal is detected by transmitting a radar signal to a subject to be measured, and receiving a signal reflected from the subject to be measured which include breathing and heartbeat signals.

In addition, an impulse-radio ultra wide band (IR-UWB) radar may be used in the present invention as an embodiment. Or course, a radar of the present invention is not limited to an IR-UWB radar, according to an embodiment, various radars may be used for measuring a bio-signal from a subject to be measured.

Generally, motions due to breathing are relatively larger than motions due to heartbeat, and thus detecting a heartbeat signal is not easy due to a harmonic component and noise of a frequency component due to breathing. Particularly, a harmonic component has a significant size value by being added to external noise, and thus becomes an obstacle when detecting a heartbeat signal.

Accordingly, in the present invention, as shown in FIG. 2, a heartbeat signal is detected from a reflected signal including both breathing and heartbeat signals of a subject to be measured by using two radars 10 and 20. Herein, a method is provided where accuracy is improved while detecting a real-time heartbeat signal in a time axis rather than converting the reflected signal into a frequency domain.

In detail, a first radar (hereinafter, referred as “long distance radar”) 10 is disposed at a distance (hereinafter, referred as “long distance”) such that a heartbeat signal of a subject to be measured is not measured but a breathing signal is measured, and a second radar (hereinafter, referred as “short distance radar”) 20 is disposed at a distance (hereinafter, referred as “short distance”) such that both breathing and heartbeat signals of the subject to be measured are measured.

Herein, “the long distance” and “the short distance” may be variably set according to an embodiment such as a physical condition of a subject to be measured, a bio-signal measuring environment, etc. In an embodiment of the present invention, “the long distance” and “the short distance” are not specified.

Meanwhile, the bio-signal measuring apparatus 100 synchronizes a breathing signal measured by the long distance radar 10 (hereinafter, referred as “long distance signal”) and both breathing and heartbeat signals measured by the short distance radar 20 (hereinafter, referred as “short distance signal”) by approximating the long distance signal to the short distance signal, and detects a heartbeat signal from the short distance signal including both breathing and heartbeat signals by calculating a difference between the two signals.

As above, the present invention detects a heartbeat signal in a time axis rather than a frequency domain. Accordingly, a heartbeat rate is measured, and as well as, a heartbeat pulse is monitored in real-time, and thus a real-time bio-signal of a subject to be measured is remotely monitored in a non-contact manner.

Hereinafter, a configuration of the bio-signal measuring apparatus 100 will be described in detail with reference to FIG. 3.

FIG. 3 is a view of a block diagram showing a configuration of the bio-signal measuring apparatus according to an embodiment of the present invention.

The bio-signal measuring apparatus 100 according to an embodiment of the present invention may include a long distance radar signal obtaining unit 110, a short distance radar signal obtaining unit 120, a signal synchronizing unit 130, a bio-signal detecting unit 140, a control unit 150, and a storing unit 160.

For reference, the bio-signal measuring apparatus 100 may be provided separate from the long distance radar 10 and the short distance radar 20, or the long distance radar 10 and the short distance radar 20 may be included in the bio-signal measuring apparatus 100.

Describing each component of the bio-signal measure apparatus 100, the long distance radar signal obtaining unit 110 may obtain a long distance signal S₁ of the long distance radar 10 which is reflected from a subject to be measured, that is, a breathing signal of the subject to be measured, and the obtained signal is represented as [Formula 1] below.

S₁=S_{B1}+N₁ [Formula 1]

Herein, S_{B1} represents a breathing signal, and N₁ represents noise.

Meanwhile, the short distance radar signal obtaining unit 120 may obtain a short distance signal S₂ of the short distance radar 20 which is reflected from the subject to be measured, that is, both breathing and heartbeat signals of the subject to be measured, and the obtained signal is represented as [Formula 2] below.

S₂=S_{B2}+S_{H2}+N₂ [Formula 2]

Herein, S_{B2} represents a breathing signal, S_{B2} represents a heartbeat signal, and N₂ represents noise.

Meanwhile, the signal synchronizing unit 130 may synchronize the long distance signal S₁ and the short distance signal S₂ by generating a signal obtained by approximating the long distance signal S₁ to the short distance signal S₂.

Herein, “synchronize” means approximating a period and a phase of two signals for obtaining a difference signal between two signals.

For the same, the signal synchronizing unit 130 may generate a signal Ŝ₂ obtained by approximating the long distance signal S₁ to the short distance signal S₂ by using a least mean squares (LMS) filter or a projection method, and the signal is represented as [Formula 3] below.

Ŝ₂=f(S₁)=Ŝ_{B2}+N′₁. [Formula 3]

Herein, Ŝ_{B2} represents a signal obtained by approximating to a breathing signal Ŝ_{B2} of the short distance signal S₂, and N₁′ represents noise.

Meanwhile, the bio-signal detecting unit 140 may detect a heartbeat signal by using the approximated signal Ŝ₂ generated in the signal synchronizing unit 130 and the actual short distance signal S₂.

In other words, a difference signal S_{H} may be obtained by subtracting the approximated signal Ŝ₂ from the actual short distance signal S₂, and the difference signal S_{H} is a heartbeat signal obtained by removing a breathing signal from the actual short distance signal S₂.

The difference signal is represented as [Formula 4] below.

S_{H}=S₂−Ŝ₂=(S_{B2}−Ŝ_{B2})+S_{H2}+N. [Formula 4]

Herein, S_{B2}−ŜS_{B2} is a breathing signal, and may be calculated as 0 since the same is a value that is approximated from each other (≈0). As a result, a signal S_{H2} according to heartbeat and noise N remain.

The same is represented as [Formula 5] below.

S_{H}=S_{H2}+N

Herein, S_{H2} represents a heartbeat signal of the short distance signal S₂, and N represents noise.

Meanwhile, the control unit 150 may control such that the components of the bio-signal measuring apparatus 100, for example, the long distance radar signal obtaining unit 110, the short distance radar signal obtaining unit 120, the signal synchronizing unit 130, and the bio-signal detecting unit 140 perform the above-mentioned operations to measure a bio-signal, and control the storing unit 160 that will be described later.

Meanwhile, the storing unit 160 may store algorithm for controlling the components of the bio-signal measuring apparatus 100 by the control unit 150, and various types of data are used and generated while controlling the same.

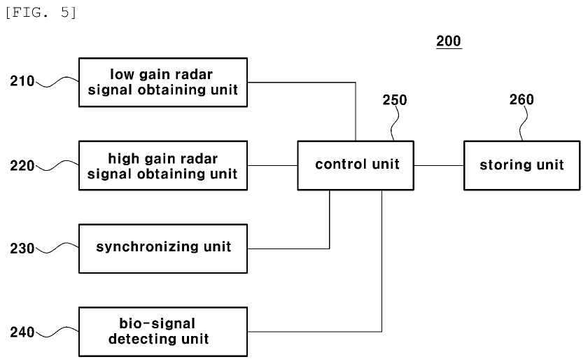

FIG. 4 is a view showing a configuration of a system for measuring a bio-signal by using a radar according to another embodiment of the present invention.

A system for measuring a bio-signal by using a radar according to another embodiment of the present invention may include a radar module 30 and a bio-signal measuring apparatus 200.

For reference, in an embodiment of FIG. 2, “distances” of two radars, for example, a long distance radar 10 and a short distance radar 20, which are different from each other are variably set on the basis of a subject to be measured such that the long distance radar measures a breathing signal and the short distance radar 20 measures both breathing and heartbeat signals.

However, in an embodiment of FIG. 4, two radars having gains different from each other constitute a single radar module 30.

In other words, a breathing signal is measured by using a first radar 31 having a gain relatively lower than the other radar (hereinafter, referred as “low gain radar”), and both breathing and heartbeat signals are measured by using a second radar 32 having a gain relatively high than the other radar (hereinafter, referred as “high gain radar”).

Herein, “the low gain” means that a gain is set such that a heartbeat signal of a subject to be measured is not measured but a breathing signal is measured, and “the high gain” means that a gain in set such that both breathing and heartbeat signals of the subject to be measured are measured.

Accordingly, a low gain or a high gain may be differently set according to a physical condition of a subject to be measured and a bio-signal measuring environment. In an embodiment of the present invention, a low gain and a high gain are not specified.

For reference, in an embodiment of FIG. 4, the low gain radar 31 and the high gain radar 32 constitute a single radar module 30. In another embodiment, the low gain radar 31 and the high gain radar 32 may be separately provided. Herein, a breathing signal or both breathing and heartbeat signals are measured according to a radar gain, and thus distances of the radars from a subject to be measured are identical.

Meanwhile, the bio-signal measuring apparatus 200 synchronizes a breathing signal measured by the low gain radar 31 (hereinafter, referred as “low gain signal”), and breathing and both heartbeat signals measured by the high gain radar 32 (hereinafter, referred as “high gain signal”) by approximating the signals, and detects a heartbeat signal by calculating a difference therebetween, that is, determines the heartbeat signal from the high gain signal.

Accordingly, the bio-signal measuring apparatus 200 detects a real-time heartbeat signal in a time axis rather than a frequency domain as the bio-signal measuring apparatus 100 of FIG. 2.

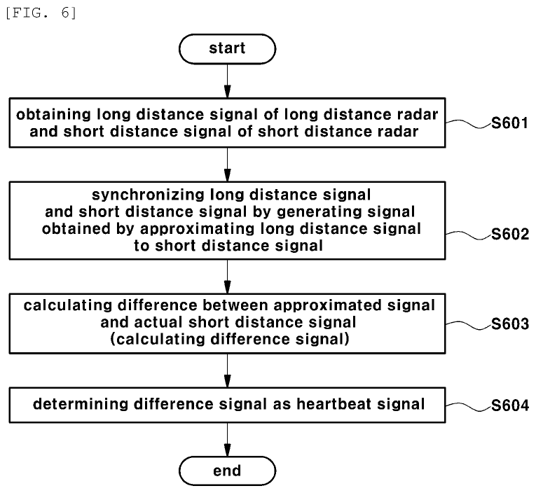

FIG. 5 is a view showing a block diagram of a bio-signal measuring apparatus according to another embodiment of the present invention.

The bio-signal measuring apparatus 200 according to another embodiment of the present invention may include a low gain radar signal obtaining unit 210, a high gain radar signal obtaining unit 220, a signal synchronizing unit 230, a bio-signal detecting unit 240, a control unit 250, and a storing unit 260.

For reference, the bio-signal measuring apparatus 200 may be provided separate from the radar module 30, or the radar module 30 may be included in the bio-signal measuring apparatus 200. Of course, the low gain radar 31 and the high gain radar 32 may not constitute a single radar module 30, and may be separately disposed.

Describing each component, the low gain radar signal obtaining unit 210 may obtain a low gain signal of the low gain radar 31, that is, a breathing signal of a subject to be measured, and the high gain radar signal obtaining unit 220 may obtain a high gain signal of the high gain radar 32, that is, both breathing and heartbeat signals of the subject to be measured.

Meanwhile, the signal synchronizing unit 230 may synchronize the low gain signal and the high gain signal by generating a signal obtained by approximating the low gain signal to the high gain signal, and the bio-signal detecting unit 240 may detect a heartbeat signal by calculating a difference between the approximated signal generated in the signal synchronizing unit 230 and the actual high gain signal.

Meanwhile, the control unit 250 may control such that the components of the bio-signal measure apparatus 200, for example, the low gain radar signal obtaining unit 210, the high gain radar signal obtaining unit 220, the signal synchronizing unit 230, and the bio-signal detecting unit 240 perform the above-mentioned operations to measure a bio-signal, and control the storing unit 260 that will be described later.

Meanwhile, the storing unit 260 may store algorithm for controlling the components of the bio-signal measuring apparatus 200 by the control unit 250, and various types of data used and generated while controlling the same.

The bio-signal measuring apparatus 200 of FIG. 5 differs from the bio-signal measuring apparatus 100 of FIG. 2 in that whether radars disposed in distances different from each other are used, or radars having gains different from each other are used.

Accordingly, the bio-signal measuring apparatus 200 of FIG. 5 detects a real-time heartbeat signal in a time axis rather than a frequency domain by using [Formula 1] to [Formula 5] described when describing the bio-signal measuring apparatus 100 of FIG. 2.

FIG. 6 is a view of a flowchart showing a process of measuring a bio-signal by using a radar according to an embodiment of the present invention.

A flowchart of FIG. 6 may be performed by the long distance radar 10, the short distance radar 20, and the bio-signal measuring apparatus 100 of FIG. 2.

Hereinafter, the flowchart of FIG. 6 will be described by using the bio-signal measuring apparatus 100.

In S601, the bio-signal measuring apparatus 100 obtains a long distance signal of the long distance radar 10 and a short distance signal of the short distance radar 20.

Herein, the long distance signal is a breathing signal of a subject to be measured, and the short distance signal is both breathing and heartbeat signals of the subject to be measured.

After S601, in S602, the bio-signal measuring apparatus 100 synchronizes the long distance signal and the short distance signal by generating a signal obtained by approximating the long distance signal including the breathing signal to the short distance signal.

After S602, in S603, the bio-signal measuring apparatus 100 calculates a difference signal between the approximated signal and the actual short distance signal.

After S603, in S604, the bio-signal measuring apparatus 100 determines a heartbeat signal that is the difference signal calculated in S603.

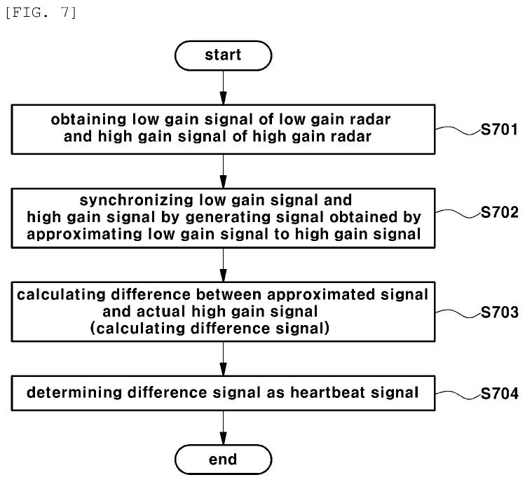

FIG. 7 is a view of a flowchart showing a process of measuring a bio-signal by using a radar according to another embodiment of the present invention.

A flowchart of FIG. 7 may be performed by the radar module 30 and the bio-signal measuring apparatus 200 of FIG. 4.

Hereinafter, the flowchart of FIG. 7 will be described by using the bio-signal measuring apparatus 200.

In S701, the bio-signal measuring apparatus 200 obtains a low gain signal of the low gain radar 31 and a high gain signal of the high gain radar 31.

Herein, the low gain signal is a breathing signal of a subject to be measured, and the high gain signal is both breathing and heartbeat signals of the subject to be measured.

After S701, in S702, the bio-signal measuring apparatus 200 synchronizes the low gain signal and the high gain signal by generating a signal obtained by approximating the low gain signal including the breathing signal to the high gain signal.

After S702, in S703, the bio-signal measuring apparatus 200 calculates a difference signal between the approximated signal and the actual high gain signal.

After S703, in S704, the bio-signal measuring apparatus determines a heartbeat signal that is the difference signal calculated in S703.

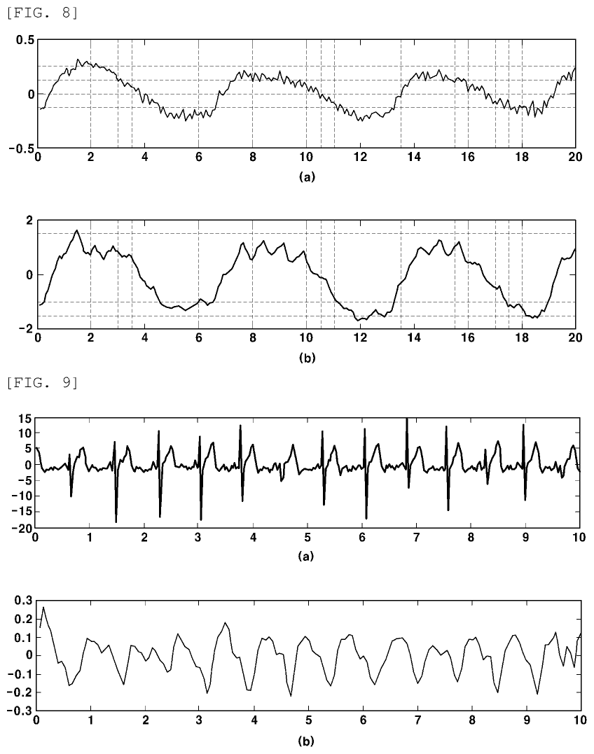

FIG. 8 is a view showing an experiment result of measuring a bio-signal by using a radar according to an embodiment of the present invention.

In FIG. 8, 8(a) shows a graph of a long distance signal of the long distance radar 10, and 8(b) shows a graph of a short distance signal of the short distance radar 20.

For reference, graphs for a low gain signal and a high gain signal of the low gain radar 31 and the high gain radar 32 will be identical to FIGS. 8(a) and 8(b), respectively.

Comparing graphs of FIGS. 8(a) and 8(b), characteristics in period are similar but in phases are different.

Accordingly, the bio-signal measuring apparatus 100 or 200 may synchronize two signals by approximating the phase difference by using an LMS filter or a projection method.

FIG. 9 is a view showing an experiment result of measuring a bio-signal by using a radar according to another embodiment of the present invention.

In FIG. 9, 9(a) shows a waveform obtained by measuring an electrical signal according to an actual heartbeat signal by using an electrocardiogram (ECG) sensor, and 9(b) shows a waveform measured by using the bio-signal measuring apparatus 100 or 200 of the present invention.

Comparing 9(a) and 9 (b), periods of the two waveforms almost coincide.

Accordingly, by using the bio-signal measuring apparatus 100 or 200 of the present invention and a bio-signal measuring method using the same, a heartbeat signal can be accurately detected from signal breathing and heartbeat signals.

The methods according to the above-described embodiments may be recorded in non-transitory computer-readable media including program instructions to implement various operations embodied by a computer.

The media may also include, alone or in combination with the program instructions, data files, data structures, and the like.

The program instructions recorded on the media may be those specially designed and constructed for the purposes of the embodiments, or they may be of the kind well-known and available to those having skill in the computer software arts.

Examples of non-transitory computer-readable media include magnetic media such as hard disks, floppy disks, and magnetic tape; optical media such as CD ROM disks and DVDs; magneto-optical media such as optical discs; and hardware devices that are specially configured to store and perform program instructions, such as read-only memory (ROM), random access memory (RAM), flash memory, and the like.

Examples of program instructions include both machine code, such as produced by a compiler, and files containing higher level code that may be executed by the computer using an interpreter.

The described hardware devices may be configured to act as one or more software modules in order to perform the operations of the above-described embodiments, or vice versa.

### INDUSTRIAL APPLICABILITY

Although the present invention has been described in terms of specific items such as detailed components as well as the limited embodiments and the drawings, they are only provided to help general understanding of the invention, and the present invention is not limited to the above embodiments. It will be appreciated by those skilled in the art that various modifications and changes may be made from the above description.

Therefore, the spirit of the present invention shall not be limited to the above-described embodiments, and the entire scope of the appended claims and their equivalents will fall within the scope and spirit of the invention.

1. An apparatus for measuring a bio-signal by using a radar, the apparatus comprising:
a first signal obtaining unit obtaining a first signal including a first bio-signal of a subject to be measured from a first radar;
a second signal obtaining unit obtaining a second signal including both of first bio-signal and a second bio-signal of the subject to be measured from a second radar;
a signal synchronizing unit synchronizing the first signal and the second signal; and
a bio-signal detecting unit calculating a difference between the synchronized first and second signals, and determining the second bio-signal by removing the first bio-signal from the second signal.

2. The apparatus of claim 1, wherein the first radar is disposed at a set distance such that the first bio-signal is measured from the subject to be measured, and the second radar is disposed at a set distance such that both of the first bio-signal and the second bio-signal are measured.

3. The apparatus of claim 2, wherein the second radar is disposed closer to the subject than the first radar.

4. The apparatus of claim 1, wherein the first radar has a set gain such that the first bio-signal is measured from the subject to be measured, and the second radar has a set gain such that both of the first bio-signal and the second bio-signal are measured from the subject to be measured.

5. The apparatus of claim 4, wherein the set gain of the second radar is high than the set gain first radar.

6. The apparatus of claim 1, wherein the first bio-signal is a breathing signal of the subject to be measured, and the second bio-signal is a heartbeat signal of the subject to be measured.

7. The apparatus of claim 1, wherein the signal synchronizing unit synchronizes the first signal and the second signal by approximating a period and a phase of the first signal to the second signal.

8. The apparatus of claim 7, wherein the signal synchronizing unit approximates the period and the phase of the first signal to the second signal by using a least mean squares (LMS) filter or a projection method.

9. The apparatus of claim 1, wherein the first radar and the second radar constitute a single radar module.

10. A method of measuring a bio-signal, wherein a bio-signal measuring apparatus measures a bio-signal by using a radar, the method comprising:
(a) obtaining a first signal including a first bio-signal of a subject to be measured from a first radar, and obtaining a second signal including both of the first bio-signal and a second bio-signal of the subject to be measured from a second radar;
(b) synchronizing the first signal and the second signal; and
(c) calculating a difference between the synchronized first signal and second signals, and determining the second bio-signal by removing the first bio-signal from the second signal.

11. The method of claim 10, wherein the first radar is disposed at a set distance such that the first bio-signal is measured from the subject to be measured, and the second radar is disposed in a set distance such that both of the first bio-signal and the second bio-signal are measured.

12. The method of claim 10, wherein the first radar has a set gain such that the first bio-signal is measured from the subject to be measured, and the second radar has a set gain such that both of the first bio-signal and the second bio-signal are measured from the subject to be measured.

13. The method of claim 10, wherein the first bio-signal is a breathing signal of the subject to be measured, and the second bio-signal is a heartbeat signal of the subject to be measured.

14. The method of claim 10, wherein, at step (b), the first signal and the second signal are synchronized by approximating a period and a phase of the first signal to the second signal.

15. A computer program stored in a recoding medium, wherein the computer program includes a series of instructions for performing the method according to claim 10.
